(19) United States
(12) Patent Application Publication
(10) Pub. No.: US 2020/0094041 A1
(43) Pub. Date: Mar. 26, 2020

(54) Wearable Electromyography Biofeedback Therapeutic Device

(71) Applicant: SHENZHEN XFT MEDICAL LIMITED, Shenzhen (CN)

(72) Inventor: Tianwei Song, Shenzhen (CN)

(21) Appl. No.: 16/467,469

(22) PCT Filed: Nov. 7, 2017
(86) PCT No.: PCT/CN2017/109645

§ 371 Date: Jun. 6, 2019

(30) Foreign Application Priority Data
May 27, 2017 (CN) 201720615149X

(51) Int. Cl. A61N 1/04; A61B 5/00; A61N 1/36; A61B 5/0492

(52) U.S. Cl. CPC A61N 1/0484; A61B 5/486; A61N 1/0452; A61N 1/3603; A61B 5/0492; A61B 5/681; A61N 1/36003

(57) ABSTRACT

Provided is a wearable electromyography biofeedback therapeutic device, including a host and a wristband, wherein the wristband includes a fixed platform for fixing the host and a bandage for wearing, the bandage is fixed to one side of the fixed platform, and the side where the fixed platform is opposite to the bandage is provided with a buckle position corresponding to the bandage; the fixed platform is provided with a first through slot matching the host; a first electrode is arranged at the bottom of the host, a button is arranged at the upper portion of the host, and a PCBA is provided inside the host, and the first electrode and the button are electrically connected to the PCBA. The wearable electromyography biofeedback therapeutic device integrates the electrode directly with the host, and the electrode is directly stimulated to the wrist portion when worn on the wrist of the patient.

### TECHNICAL FIELD

The present disclosure relates to medical therapeutic equipment, particularly to a wearable myoelectricity (electromyography) biofeedback therapeutic apparatus (device).

### BACKGROUND ART

Latest discoveries in current society and statistical data from World Health Organization indicate that China is a nation where cerebral stroke is prevalent, with up to six million of surviving cerebral stroke patients, among which 4.5 million are disabled. The startling data tells us that cerebral stroke not only is a major killer harming human beings, but also has extremely high morbidity and mortality. In this regard, rehabilitation therapy for cerebral stroke patients is put in the first place in clinical medical science.

In recent years, the human body myoelectricity biofeedback functional electrical stimulation therapeutic regimen is put into wide use, which belongs to an medical electronic product for rehabilitation physical therapy, for repair and rehabilitation training therapy of motor functions of limbs and muscles of paralyzed patients caused by diseases such as cerebral stroke.

For existing myoelectricity biofeedback therapeutic apparatus, a host and an electrode are separated, and the electrode needs to be connected with the host through a connection line, which is tedious to operate and inconvenient to use.

### SUMMARY

A wearable myoelectricity biofeedback therapeutic apparatus provided in the present disclosure solves one or more of the existing technical problems.

The wearable myoelectricity biofeedback therapeutic apparatus provided in the present disclosure includes a host and a wristband;

the wristband includes a fixed platform for fixing the host and a bandage for wearing, the bandage is fixed at one side of the fixed platform, and the fixed platform is provided, at one side opposite to the bandage, with a buckling position corresponding to the bandage;

the fixed platform is provided with a first through slot matched with the host; and

the host is provided with a first electrode in a bottom portion, with a button in an upper portion, and with a PCBA inside, and the first electrode and the button are both electrically connected with the PCBA.

In the wearable myoelectricity biofeedback therapeutic apparatus provided in the present disclosure, the electrode is directly connected with the host in one piece, the electrode directly performs stimulation treatment to a wrist site when worn on a patient's wrist, thus the operation is simple and convenient, and meanwhile the device is convenient to carry.

In some embodiments of the present disclosure, a second electrode and a connecting line are further included, wherein the second electrode is provided at one end of the connecting line, and a side face of the host is provided with a port configured to be connected with one end of the connecting line that is not connected with the second electrode.

In this way, the second electrode, connected with the host through the connecting line, can perform stimulation treatment to other sites of a body, without taking the therapeutic apparatus from the wrist.

In some embodiments of the present disclosure, when the second electrode is connected with the host, a main control unit on the PCBA recognizes the second electrode, at which time the first electrode is automatically switched off.

In this way, when the second electrode is connected, the second electrode is used preferentially, and the first electrode and the second electrode are automatically switched, without man-made setting, thus being convenient to use.

In some embodiments of the present disclosure, the host includes a host upper shell and a host lower shell, the PCBA is fixed on the host upper shell, and the host upper shell and the host lower shell are in buckled connection;

the host upper shell is provided with a second through slot configured for allowing the button to pass therethrough, and the button is matched with a button board on the PCBA and extends through the second through slot to reach to the outside of the host.

In this way, the assembling of the PCBA and the button is facilitated.

In some embodiments of the present disclosure, the host upper shell is provided with a film sheet on one face facing away from the host lower shell, and the film sheet is provided with a third through slot configured for allowing the button to extend out therefrom.

In this way, the film sheet can protect a display screen of the host, and the button extends out of the film sheet to allow complete attachment of the film sheet with the host upper shell.

In some embodiments of the present disclosure, the fixed platform includes a fixed upper shell and a fixed lower shell, and the fixed upper shell and the fixed lower shell are injection-molded in one piece;

the fixed upper shell is made of a hard rubber material, and the fixed lower shell is made of a soft rubber material.

In this way, using the hard rubber material to make the fixed upper shell prevents the fixed upper shell from deformation due to stretching of the bandage, ensuring that the host fixed at the fixed platform can be reliably fastened without assembling clearance appearing due to a pulling force of the bandage. As the fixed lower shell is made of the soft material, the flexibility of the soft rubber can render it more comfortable for a patient to wear.

In some embodiments of the present disclosure, a fastener incorporated with the bandage is further included, the bandage is provided with a mounting groove configured to mount the fastener, the mounting groove is provided with a first through hole, and the fastener is provided with a clamp pin capable of being clamped and fastened in the first through hole;

the bandage is provided thereon with a second through hole allowing the clamp pin to be clamped and fastened therein, and the bandage is able to be fixed by wrapping the bandage on the wrist, putting the bandage to pass through the buckling position, and then folding back the bandage to clamp the clamp pin of the fastener in the second through hole.

In this way, by annularly fastening one bandage, a patient can wear the device by himself/herself with a single hand.

In some embodiments of the present disclosure, one face of the bandage in contact with skin is provided with several bulges, and the bulges are made of a soft colloidal substance.

In this way, the bulges made of the soft colloidal substance generates flexible friction when contacting with the skin, which prevents displacement of the host to stagger an operating part of the first electrode in position and affect the therapeutic effect.

In some embodiments of the present disclosure, the PCBA includes an MCU main control circuit, a power supply circuit, a button circuit, an electrical stimulation circuit, a myoelectricity detection circuit, and an OLED display circuit

In this way, operation of the whole host can be achieved.

### BRIEF DESCRIPTION OF DRAWINGS

FIG. 1 is a structural schematic diagram of a wearable myoelectricity biofeedback therapeutic apparatus in an embodiment provided in the present disclosure;

FIG. 2 is an exploded view of the wearable myoelectricity biofeedback therapeutic apparatus in an embodiment provided in the present disclosure;

FIG. 3 is a schematic diagram of positioning a first electrode with the wearable myoelectricity biofeedback therapeutic apparatus in an embodiment provided in the present disclosure being worn on a wrist;

FIG. 4 is a structural schematic diagram showing connection of a host and second electrodes of the wearable myoelectricity biofeedback therapeutic apparatus in an embodiment provided in the present disclosure;

FIG. 5 is a circuit diagram when merely the first electrode is used in the wearable myoelectricity biofeedback therapeutic apparatus in an embodiment provided in the present disclosure; and

FIG. 6 is a circuit diagram when the second electrodes are used in the wearable myoelectricity biofeedback therapeutic apparatus in an embodiment provided in the present disclosure.

### DETAILED DESCRIPTION OF EMBODIMENTS

The present disclosure is further described in detail below in combination with accompanying drawings in the specification.

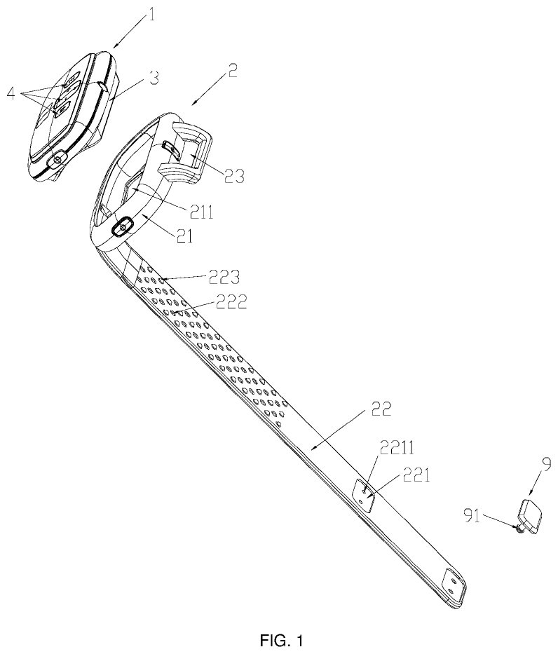

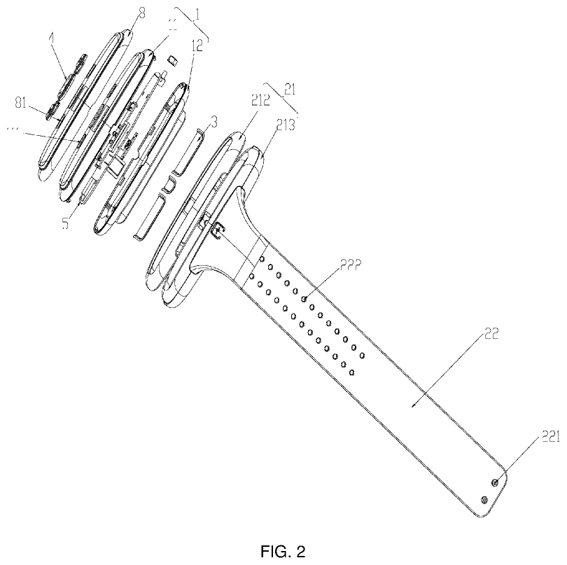

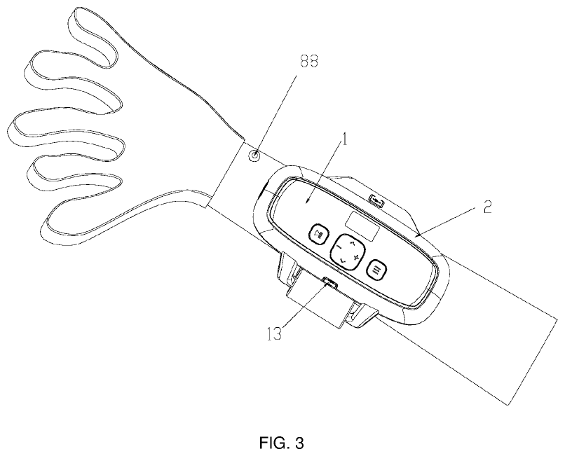

Referring to FIG. 1 to FIG. 3, a wearable myoelectricity biofeedback therapeutic apparatus includes a host 1 and a wristband 2, wherein the wristband 2 includes a fixed platform 21 for fixing the host 1 and a bandage 22 for wearing. The bandage 22 is fixed at one side of the fixed platform 21, and the fixed platform 21 is provided, at one side opposite to the bandage 22, with a buckling position 23 corresponding to the bandage 22, the fixed platform 21 is provided with a first through slot 211 matched with the host 1, the host 1 is provided with a first electrode 3 in a bottom portion, with a button 4 in an upper portion, and with a PCBA 5 inside. The first electrode 3 and the button 4 are both electrically connected with the PCBA 5.

When in use, after the host 1 is placed with one fingerbreadth to a wrist joint 88, the wristband 2 is put through the buckling position 23 to fix the therapeutic apparatus on the wrist, achieving positioning of the first electrode 3. At this position, uplift rehabilitation therapy of the wrist can be realized.

Specifically, the host 1 includes a host upper shell 11 and a host lower shell 12, the PCBA 5 is fixed on the host upper shell 11, and the host upper shell 11 and the host lower shell 12 are in buckled connection; the host upper shell 11 is provided with a second through slot 111 configured for allowing the button 4 to pass therethrough, and the button 4 is matched with a button board on the PCBA 5 and extends through the second through slot 111 to reach the outside of the host 1. In this way, an outer shell of the host 1 is divided into two parts, facilitating the assembling of the PCBA 5 inside and the button 4.

In the above, the host upper shell 11 is provided with a film sheet 8 on one face facing away from the host lower shell 12, and the film sheet 8 is provided with a third through slot 81 configured for allowing the button 4 to extend out therefrom. In this way, the film sheet 8 can be allowed to stay away from the button 4 and closely attach to the host upper shell 11, thus protecting the host upper shell 11. In practical manufacturing, different colors and patterns can be screen-printed on the film sheet 8, such that the host upper shell 11 is colorful.

Specifically, the fixed platform 21 includes a fixed upper shell 212 and a fixed lower shell 213, wherein the fixed upper shell 212 and the fixed lower shell 213 are injection-molded in one piece, the fixed upper shell 212 is made of a hard rubber material, and the fixed lower shell 213 is made of a soft rubber material.

In this way, using the hard rubber material to make the fixed upper shell 212 prevents the fixed upper shell from deformation due to stretching of the bandage 22, ensuring that the host 1 fixed at the fixed platform 21 can be reliably fastened without assembling clearance appearing due to a pulling force of the bandage 22; as the fixed lower shell 213 is made of the soft rubber material, the flexibility of the soft rubber can render it more comfortable for a patient to wear.

Further, a fastener 9 incorporated with the bandage 22 is further included, the bandage 22 is provided with a mounting groove 221 configured to mount the fastener 9, the mounting groove 221 is provided with a first through hole 2211, and the fastener 9 is provided with a clamp pin 91 capable of being clamped and fastened in the first through hole 2211. In the present embodiment, there may be multiple mounting grooves 221, and the fastener 9 is correspondingly mounted in the mounting groove 221 according to the size of the wrist, so as to adjust the bandage 22.

The bandage 22 is provided thereon with a second through hole 222 allowing the clamp pin 91 to be clamped and fastened therein, and the bandage 22 can be fixed by wrapping the bandage on the wrist, putting the bandage to pass through the buckling position 23, and then folding back the bandage to clamp the clamp pin 91 of the fastener 9 in the second through hole 222. With the fastening manner of folding back one bandage 22, single-hand self-wearing is achievable, and the operation is simple and convenient.

In the above, one face of the bandage 22 in contact with skin is provided with several bulges 223, and the bulges 223 are made of a soft colloidal substance. The soft bulges 223 can generate flexible friction with the skin, which not only creates no discomfort feeling to the skin, but also can prevent rotation of the host 1 on the wrist to stagger the first electrode 3 and a treated site in position and affect the therapeutic effect.

Further, the PCBA 5 includes an MCU main control circuit, a power supply circuit, a button circuit, an electrical stimulation circuit, a myoelectricity detection circuit, and an OLED display circuit.

When the stimulation therapy needs to be performed for other sites of a body, the first electrode 3 provided in the bottom portion of the host 1 is limited in position, therefore, the device also needs to be able to be flexibly used for treatment of other sites of the body when the host 1 is worn on the wrist.

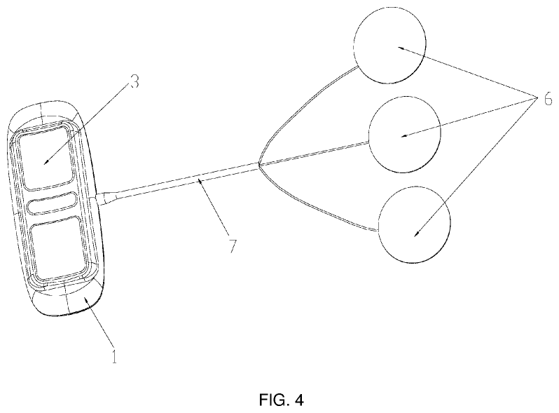

Referring to FIG. 4, further, second electrodes 6 and a connecting line 7 are further included, wherein the second electrodes 6 are provided at one end of the connecting line 7, and a side face of the host 1 is provided with a port 13 configured to be connected with one end of the connecting line 7 that is not connected with the second electrodes 6. Thus, the second electrodes 6 can perform treatment for other sites of the body, and the length of the connecting line 7 can be selected according to practical requirements.

In the above, when the second electrodes 6 are connected with the host 1, the main control unit on the PCBA 5 recognizes the second electrodes 6, at which time the first electrode 3 is automatically switched off. In this way, when the second electrodes 6 are connected, the first electrode 3 and the second electrodes 6 share one circuit, at which time, the second electrodes 6 are used preferentially.

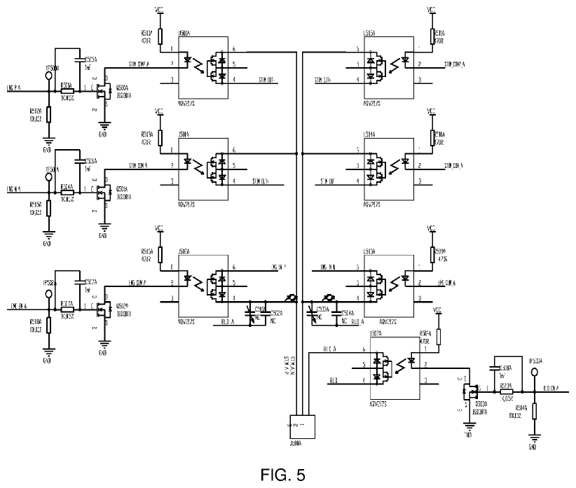

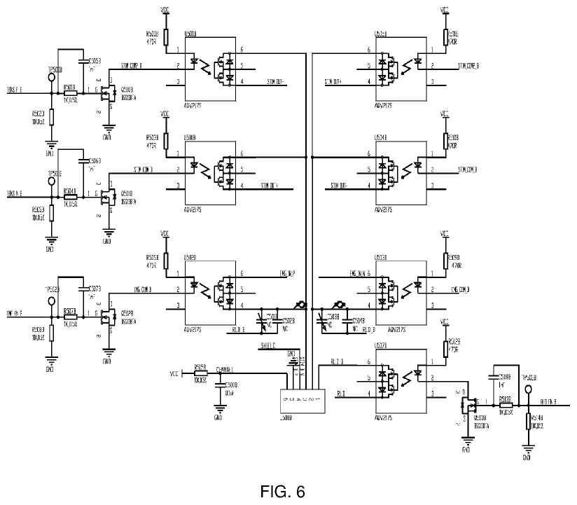

Referring to FIG. 5 and FIG. 6, an implementation principle is as follows: two contact pins inside a plug of the connecting line 7 connected with the second electrodes 6 are short-circuited, after the second electrodes 6 are connected with the port 13 of the host 1, one of the contact pins is connected with a detection pin (a sixth pin of J500B) of a single chip, the other contact pin is connected with “ground” (a fifth pin of J500B), and since the two contact pins are short-circuited, the single chip can identify insertion of an external electrode.

In the above, J500A is an internal electrode terminal, J500B is an external electrode terminal, the sixth pin, CHANNEL of J500B recognizes and detects connection of the external electrode. When no external electrode is connected, the sixth pin, CHANNEL of J500B is at a high level, I/O control ports of the single chip, ENS_P_A, TENS_N_A, EMG_EN_A, and RLD_EN_A, output a high level (meanwhile, TENS_P_B, TENS_N_B, EMG_EN_B, and RLD_EN_B output a low level), 7 photoelectric couplers, U500A, U501A, U502A, U503A, U504A, U505A, and U507A, are all switched on, (meanwhile, 7 photoelectric couplers, U500B, U501B, U502B, U503B, U504B, U505B, and U507B are all switched off), and 5 paths of signals, STIM_OUT+, STIM_OUT−, EMG_IN_P, EMG_IN_N, and RLD, are output to the internal electrode terminal J500A;

After the external electrode is connected, the sixth pin, CHANNEL of J500B is at a low level, the I/O control ports of the single chip, TENS_P_B, TENS_N_B, EMG_EN_B, and RLD_EN_B, output a high level (meanwhile, TENS_P_A, TENS_N_A, EMG_EN_A, and RLD_EN_A output a low level), 7 photoelectric couplers, U500B, U501B, U502B, U503B, U504B, U505B, and U507B, are all switched on, (meanwhile, 7 photoelectric couplers, U500A, U501A, U502A, U503A, U504A, U505A, and U507A, are all switched off), and 5 paths of signals, STIM_OUT+, STIM_OUT−, EMG_IN_P, EMG_IN_N, and RLD, are output to the external electrode terminal J500B.

There are merely some embodiments of the present disclosure in the above. A person ordinarily skilled in the art still can make several modifications and improvements without departing from the inventive concept of the present disclosure, all of which fall within the scope of protection of the present disclosure.

1. Wearable myoelectricity biofeedback therapeutic apparatus, comprising a host and a wristband
wherein the wristband comprises a fixed platform for fixing the host and a bandage for wearing, the bandage is fixed at one side of the fixed platform and the fixed platform is provided, at one side opposite to the bandage with a buckling position corresponding to the bandage
the fixed platform is provided with a first through slot matched with the host and
the host is provided with a first electrode in a bottom portion, with a button in an upper portion, and with a PCBA inside, and the first electrode and the button are both electrically connected with the PCBA

2. The wearable myoelectricity biofeedback therapeutic apparatus according to claim 1, further comprising a second electrode and a connecting line wherein the second electrode is provided at one end of the connecting line and a side face of the host is provided with a port configured to be connected with one end of the connecting line that is not connected with the second electrode

3. The wearable myoelectricity biofeedback therapeutic apparatus according to claim 2, wherein in a case that the second electrode is connected with the host when a main control unit on the PCBA recognizes the second electrode the first electrode is automatically switched off.

4. The wearable myoelectricity biofeedback therapeutic apparatus according to claim 1, wherein the host comprises a host upper shell and a host lower shell the PCBA is fixed on the host upper shell and the host upper shell and the host lower shell are in buckled connection;
the host upper shell is provided with a second through slot configured for allowing the button to pass therethrough, and the button is matched with a button board on the PCBA and extends through the second through slot to reach an outside of the host

5. The wearable myoelectricity biofeedback therapeutic apparatus according to claim 4, wherein the host upper shell is provided with a film sheet on one face facing away from the host lower shell and the film sheet is provided with a third through slot configured for allowing the button to extend out therefrom.

6. The wearable myoelectricity biofeedback therapeutic apparatus according to claim 1, wherein the fixed platform comprises a fixed upper shell and a fixed lower shell and the fixed upper shell and the fixed lower shell are injection-molded in one piece;
the fixed upper shell is made of a hard rubber material, and the fixed lower shell is made of a soft rubber material.

7. The wearable myoelectricity biofeedback therapeutic apparatus according to claim 1, further comprising a fastener incorporated with the bandage wherein the bandage is provided with a mounting groove configured to mount the fastener the mounting groove is provided with a first through hole and the fastener is provided with a clamp pin capable of being clamped and fastened in the first through hole;
the bandage is provided thereon with a second through hole allowing the clamp pin to be clamped and fastened therein, and the bandage is able to be fixed by: wrapping the bandage on a wrist, putting the bandage to pass through the buckling position, and then folding back the bandage to clamp the clamp pin of the fastener in the second through hole

8. The wearable myoelectricity biofeedback therapeutic apparatus according to claim 7, wherein one face of the bandage in contact with skin is provided with several bulges, and the bulges are made of a soft colloidal substance.

9. The wearable myoelectricity biofeedback therapeutic apparatus according to claim 1, wherein the PCBA comprises an MCU main control circuit, a power supply circuit, a button circuit, an electrical stimulation circuit, an myoelectricity detection circuit, and an OLED display circuit.

10. The wearable myoelectricity biofeedback therapeutic apparatus according to claim 2, wherein the PCBA comprises an MCU main control circuit, a power supply circuit, a button circuit, an electrical stimulation circuit, an myoelectricity detection circuit, and an OLED display circuit.

11. The wearable myoelectricity biofeedback therapeutic apparatus according to claim 3, wherein the PCBA comprises an MCU main control circuit, a power supply circuit, a button circuit, an electrical stimulation circuit, an myoelectricity detection circuit, and an OLED display circuit.

12. The wearable myoelectricity biofeedback therapeutic apparatus according to claim 4, wherein the PCBA comprises an MCU main control circuit, a power supply circuit, a button circuit, an electrical stimulation circuit, an myoelectricity detection circuit, and an OLED display circuit.

13. The wearable myoelectricity biofeedback therapeutic apparatus according to claim 5, wherein the PCBA comprises an MCU main control circuit, a power supply circuit, a button circuit, an electrical stimulation circuit, an myoelectricity detection circuit, and an OLED display circuit.

14. The wearable myoelectricity biofeedback therapeutic apparatus according to claim 6, wherein the PCBA comprises an MCU main control circuit, a power supply circuit, a button circuit, an electrical stimulation circuit, an myoelectricity detection circuit, and an OLED display circuit.

15. The wearable myoelectricity biofeedback therapeutic apparatus according to claim 7, wherein the PCBA comprises an MCU main control circuit, a power supply circuit, a button circuit, an electrical stimulation circuit, an myoelectricity detection circuit, and an OLED display circuit.

16. The wearable myoelectricity biofeedback therapeutic apparatus according to claim 8, wherein the PCBA comprises an MCU main control circuit, a power supply circuit, a button circuit, an electrical stimulation circuit, an myoelectricity detection circuit, and an OLED display circuit.
